(19) United States
(12) Patent Application Publication
(10) Pub. No.: US 2026/0000274 A1
(43) Pub. Date: Jan. 1, 2026

(54) ENDOSCOPE DISTAL TIP ATTACHMENT

(71) Applicant: GYRUS ACMI, INC. D/B/A OLYMPUS SURGICAL TECHNOLOGIES AMERICA, Westborough, MA (US)

(72) Inventors: Joey Magno, Dudley, MA (US); Michael E. Callaghan, Northborough, MA (US)

(21) Appl. No.: 18/880,716

(22) PCT Filed: Jul. 13, 2023
(86) PCT No.: PCT/US2023/070132

§ 371 Date: Jan. 2, 2025

Related U.S. Application Data
(60) Provisional application No. 63/368,518, filed on Jul. 15, 2022.

(51) Int. Cl. A61B 1/00

(52) U.S. Cl. CPC A61B 1/00101; A61B 1/00131

(57) ABSTRACT

An endoscope distal tip attachment is provided that can include a barrel and distal tip extending from the barrel. The barrel can have a proximal end and a distal end along with a first shoulder and a second shoulder. The first shoulder can be at a first side of the distal end and can be configured to engage with a distal end of an endoscope. The second shoulder can be at second side of the distal end opposing the distal end first side. The barrel can have a set of ribs extending away from the first shoulder and towards the barrel proximal end where the set of ribs define a cavity. The barrel can include a ring coupled with the set of ribs and extends around the cavity. The distal tip can extend from the second barrel shoulder and can be formed as a single piece with the barrel.

### PRIORITY CLAIM

This application claims the benefit of priority to U.S. Provisional Patent Application Ser. No. 63/368,518, filed Jul. 15, 2022, the contents of which are incorporated by reference.

### TECHNICAL FIELD

This document pertains generally, but not by way of limitation, to surgical devices that can be used for various surgical procedures. More specifically, but not by way of limitation, the present application relates to an attachment member for an endoscope.

### BACKGROUND

Endoscopy is a procedure that uses an endoscope to examine a patient. In an upper endoscopy, an endoscope can be passed through the mouth and throat of a patient and into the esophagus during the procedure. From there, the endoscope can pass into the stomach and small intestines of the patient. In a lower endoscopy, or colonoscopy, an endoscope called a colonoscope is passed through the anus of the patient and the physician examens the large intestine of the patient. The endoscope includes a camera to allow observation of the throat, esophagus, stomach, small intestine, and large intestine.

Issues can arise where a tip of the endoscope having a camera can come into contact with tissue surrounding an area being observed. If tissue contacts the camera, the field of view of the camera can be obscured, thereby limiting the effectiveness of the endoscopic procedure. Similarly, foreign bodies can contact the camera, again obscuring a field of view of the camera and limiting the effectiveness of the endoscopic procedure.

### SUMMARY

An approach to prevent foreign bodies from coming into contact with an end of an endoscope and potentially obscuring the field of view of a camera can include an attachment that fits at a distal end of the endoscope. The attachment can be made of a clear material. Nevertheless, problems can arise with this approach, such as with securing the attachment to the distal end of the endoscope.

Accordingly, what is needed is an attachment member for an endoscope that is capable of maintaining a field of view for a camera while at the same time preventing foreign bodies from contacting the camera. Moreover, the attachment should be capable of being secured to an end of the endoscope with minimal effort.

This document describes examples that can relate to an endoscope distal tip attachment. The endoscope distal tip attachment can include an outer barrel having a proximal end and a distal end. The outer barrel can include ribs that extend away from the distal end towards the proximal end, where the ribs can define a cavity. An inner barrel can be disposed within the cavity such that the inner barrel is within the outer barrel. The inner barrel can be configured to receive a distal end of an endoscope. In examples, the combination of the outer and inner barrels can provide a structure that can fit over a distal end of an endoscope while minimizing damage to the endoscope. The outer barrel can include a ring that extends around the cavity and is coupled with the ribs. The ring can be expandable to allow fitment of the outer and inner barrels around an endoscope and contractable to provide a snug fit around the endoscope.

The outer barrel can include a first shoulder located at the distal end against which a distal end of an endoscope disposed within the inner barrel can abut. Moreover, the outer barrel can include a second shoulder that opposes the first shoulder from which a distal tip extends. In examples, the distal tip can be unitary with the outer barrel. To further illustrate, the distal tip and the outer barrel can be formed from a single piece. The distal tip can include a wall that extends from the second shoulder of the outer barrel. The wall can define a cavity that exposes a distal end of an endoscope when the endoscope is disposed within the outer and inner barrels and abuts the first shoulder.

An advantage can include providing a distal tip that can be situated near a distal end of an endoscope in order to help minimize obstructions that can interfere with the endoscope.

Another advantage can include the ability to provide a distal tip that is unitary with an assembly that can fit over an endoscope where the distal tip only contacts a distal end of endoscope via the assembly and need not be in direct contact with the endoscope such that the distal tip does not require any type of securing to the endoscope.

Another advantage can relate to the ability to insert a distal tip over an end of an endoscope while helping avoiding or minimizing damage to the endoscope.

### BRIEF DESCRIPTION OF FIGURES

FIG. 1 illustrates an endoscope assembly having an endoscope distal tip attachment, in accordance with at least one example of the present disclosure.

FIGS. 2A and 2B show the endoscope distal tip attachment in FIG. 1 in greater detail, in accordance with at least one example of the present disclosure.

FIG. 3 illustrates an outer barrel of the endoscope distal tip attachment in FIG. 1, in accordance with at least one example of the present disclosure.

FIG. 4 illustrates a cavity formed by outer barrel ribs of the outer barrel shown with reference to FIG. 3, where an endoscope distal end is disposed within the cavity, in accordance with at least one example of the present disclosure.

FIG. 5 illustrates an outer barrel of the endoscope distal tip attachment in FIG. 1, in accordance with at least one example of the present disclosure.

FIG. 6 illustrates an inner barrel of the endoscope distal tip attachment of FIG. 1, in accordance with at least one example of the present disclosure.

FIG. 7 is a flowchart illustrating a reprocessing method for the endoscope distal attachment of FIGS. 2A and 2B, in accordance with at least one example of the present disclosure.

FIGS. 8A and 8B are an alternative example of the distal tip attachment of FIGS. 2A and 2B, in accordance with at least one example of the present disclosure.

### DETAILED DESCRIPTION

Examples of the present disclosure relate to an endoscope distal tip attachment assembly. The endoscope distal tip attachment assembly can include a barrel having a proximal end and a distal end where a first shoulder is located at the proximal end of the barrel. In addition, the barrel can include a second shoulder that opposes the first shoulder at the distal end where a distal extends from the second shoulder. The distal tip can be unitary with the barrel such that the barrel and the distal tip are single unit.

In further examples, the endoscope distal tip attachment can include an outer barrel having a proximal end and a distal end. The outer barrel can include ribs that extend away from the distal end towards the proximal end, where the ribs can define a cavity. An inner barrel can be disposed within the cavity such that the inner barrel is within the outer barrel. The inner barrel can be configured to receive a distal end of an endoscope. In examples, the combination of the outer and inner barrels can provide a structure that can fit over a distal end of an endoscope while minimizing damage to the endoscope. The outer barrel can include a ring that extends around the cavity and is coupled with the ribs. The ring can be expandable to allow fitment of the outer and inner barrels around an endoscope and contractable to provide a snug fit around the endoscope.

The outer barrel can include a first shoulder located at the distal end against which a distal end of an endoscope disposed within the inner barrel can abut. Moreover, the outer barrel can include a second shoulder that opposes the first shoulder from which a distal tip extends. In examples, the distal tip can be unitary with the outer barrel. To further illustrate, the distal tip and the outer barrel can be formed from a single piece. The distal tip can include a wall that extends from the second shoulder of the outer barrel. The wall can define a cavity that exposes a distal end of an endoscope when the endoscope is disposed within the outer and inner barrels and abuts the first shoulder.

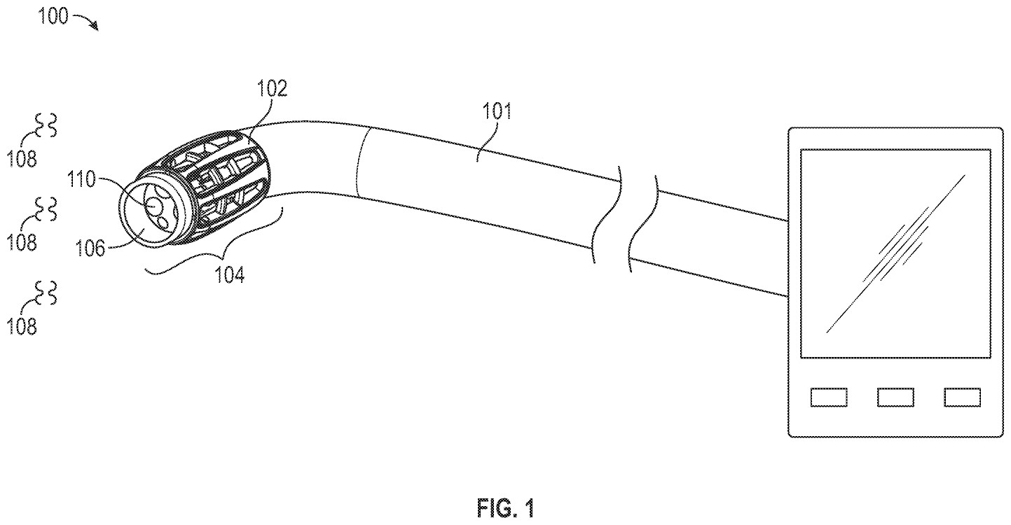

FIG. 1 shows an endoscope assembly 100 having an endoscope 101 that includes an endoscope distal tip attachment 102. The endoscope distal tip attachment 102 can be disposed at a distal end 104 of the endoscope. The endoscope distal tip attachment 102 can include a distal tip 106, which, as will described in greater detail below, can be configured to minimize foreign bodies 108 from obscuring a camera 110 of the endoscope assembly 100. While the foreign bodies 108 are shown as potentially coming into contact with and obscuring the endoscope camera 110, the distal tip 106 can function to help reduce or minimize tissue within a surgical site from contacting the endoscope camera 110.

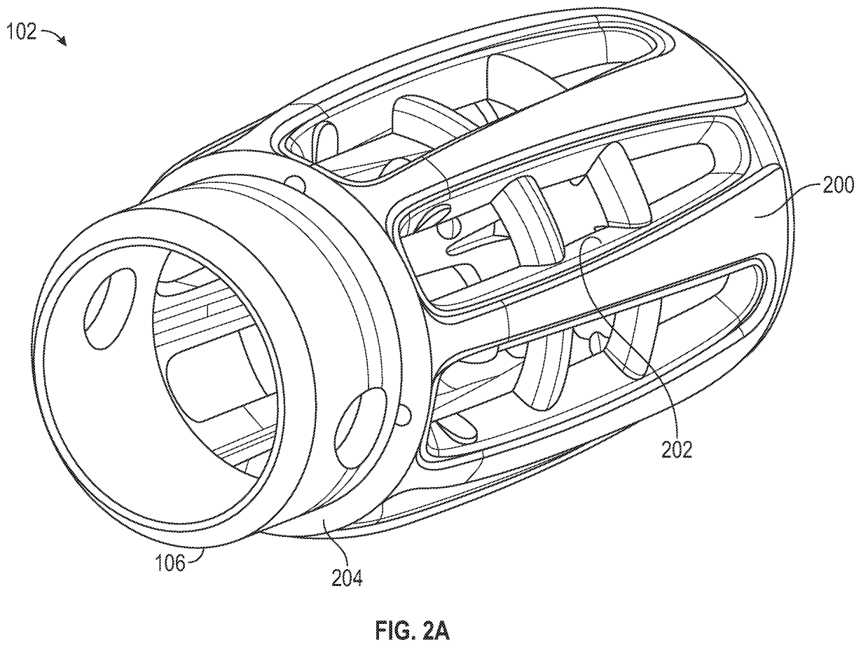

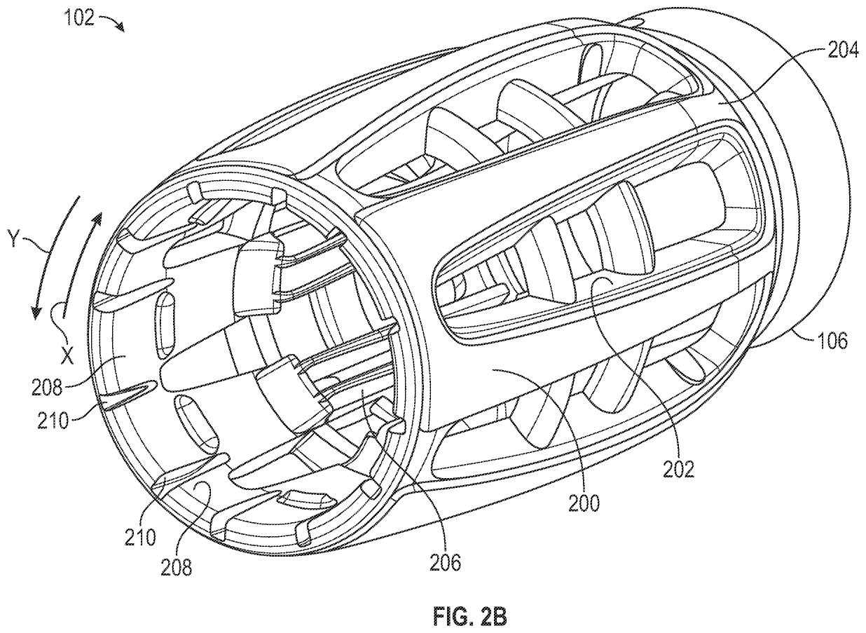

The distal tip 106 can extend from the endoscope distal tip attachment 102, as shown in greater detail with reference to FIGS. 2A and 2B. The endoscope distal tip attachment 102, which can be an assembly, can include an outer barrel 200 and an inner barrel 202 disposed within the outer barrel 200. In addition, the distal tip 106 can extend from a distal end 204 of the outer barrel 200. The inner barrel 202 can be configured to fit around the endoscope distal end 104 such as in a manner that can help reduce or minimize damage to the endoscope distal end 104.

To help minimize damage to the endoscope distal end 104, the inner barrel 202 can be formed from a material that allows for deformation of the inner barrel 202 around the endoscope distal end 104 when a practitioner places the endoscope distal tip attachment 102 over the endoscope distal end 104. To further illustrate, the inner barrel 202 can be formed from a material having a durometer shore hardness in a range of about 70-90. Types of materials that can be used can include polymers and rubbers. As such, the inner barrel 202 can expand and contract to provide a snug fit around the endoscope distal end 104.

The inner barrel 202 can include ribs 206 can be configured to engage with the endoscope distal end 104 when the endoscope distal tip attachment 102 is placed over the endoscope distal end 104. For example, since the inner barrel 202 is formed from a material that allows for deformation of the inner barrel 202, the inner barrel ribs 206 can deform to accommodate the endoscope distal end 104. The inner barrel 202 can include engagement surfaces 208 separated by gaps 210. The engagement surfaces 208 can contact the endoscope distal end 104 during placement of the endoscope distal tip attachment 102 over the endoscope distal end 104. By virtue of the gaps 210, the engagement surfaces 208 can move along directions X and Y to further allow a snug fit of the inner barrel 202 around the endoscope distal end 104.

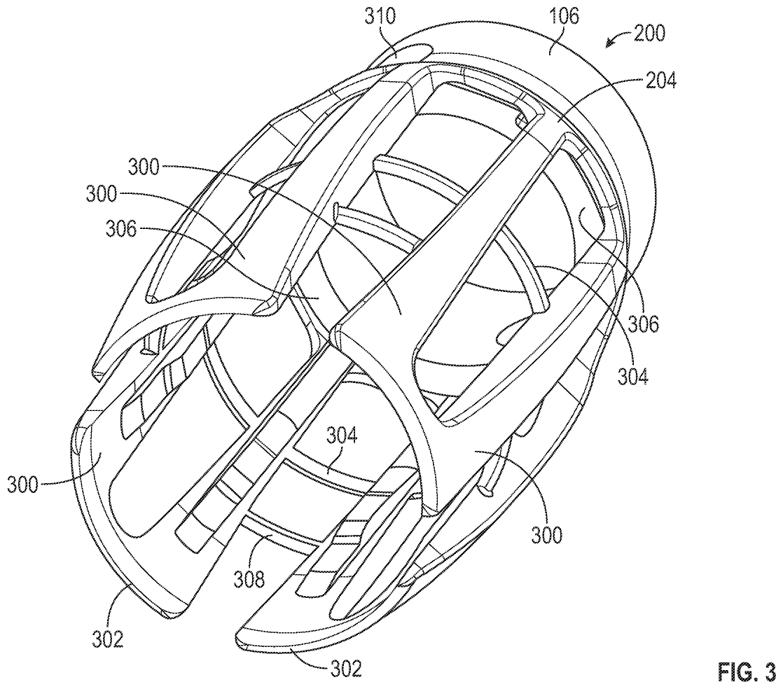

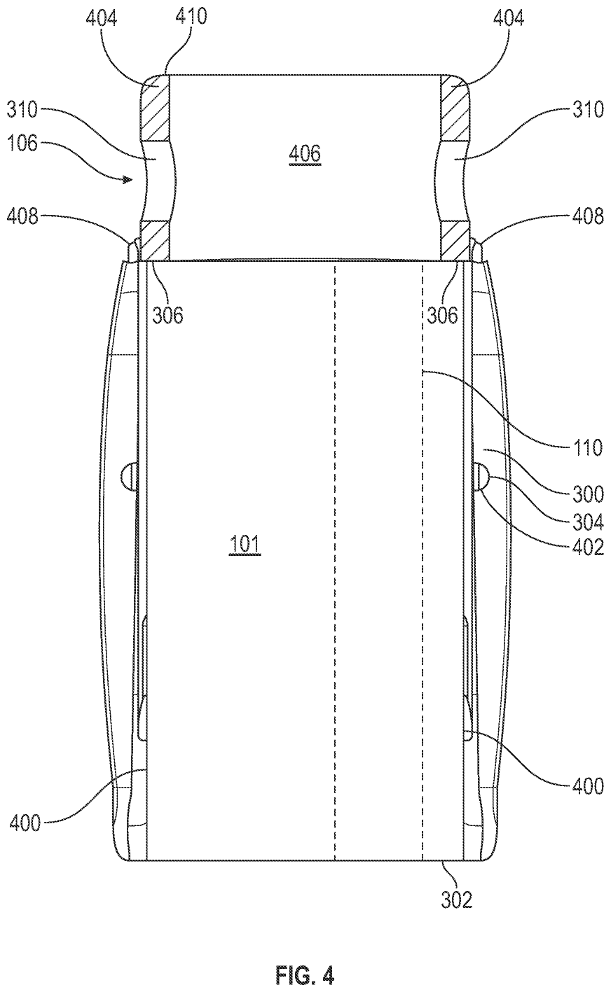

FIG. 3 shows an example of how the outer barrel 200 can help accommodate the inner barrel 202, the outer barrel 200 can include ribs 300 that extend away from the outer barrel distal end 204 towards a proximal end 302 of the outer barrel. The outer barrel ribs 300 can form a cavity 400 (FIG. 4) in which the endoscope distal end 104 can be disposed. Similar to the inner barrel 202, to help reduce or minimize damage to the endoscope distal end 104, the outer barrel ribs 300 can be formed from a material that allows for bending of the outer barrel ribs 300 when a practitioner places the endoscope distal tip attachment 102 over the endoscope distal end 104. Here, the outer barrel 200 along with the outer barrel ribs 300 can be formed from a material having a durometer shore hardness in a range of about 70-90. Types of materials that can be used can include polymers and rubbers. As such, the outer barrel ribs 300 can provide a snug fit around the endoscope distal end 104. The outer barrel 200 and the outer barrel ribs 300 can be formed of a material that has a higher durometer shore hardness in comparison to the material used for the inner barrel 202. For example, the outer barrel 200 and the outer barrel ribs 300 can be made of a material that is harder than a material for the inner barrel 202 when the inner barrel 202 is formed having a durometer shore hardness that is less than a durometer shore hardness of the outer barrel 200.

When the outer barrel ribs 300 deflect outwardly, the outer barrel 200 can include a ring 304 disposed around the cavity 400 and within the ribs 300, such as shown in FIGS. 3 and 4. The ring 304 can be disposed in a recess 402 of the outer barrel ribs 300. The ring 304 can function to move the outer barrel ribs 300 to the position shown with reference to FIG. 3 after the outer barrel ribs 300 have deflected during placement of the endoscope distal tip attachment 102 over the endoscope distal end 104. For example, the ring 304 can be formed of an elastomer, such as polyurethane, polybutadiene, neoprene and silicone, or any other type of material that allows the ring 304 to pull the outer barrel ribs 300 into the configuration shown in FIG. 3 after outward deflection during placement of the endoscope distal tip attachment 102 over the endoscope distal end 104. As such, the ring 304 can be formed of a material that is capable of expansion and contraction.

In some examples, the outer barrel distal end 204 can include a shoulder 306 at a first side 500 (FIGS. 3 & 4) of the outer barrel distal end 204. The shoulder 306 can be disposed about a periphery of the distal tip 106. For example, the shoulder 306 can be configured such that the endoscope distal end 104 abuts the shoulder 306 when the endoscope distal end 104 is disposed within the cavity 400, as shown in FIG. 4. As such, the shoulder 306 can act as a stop for the endoscope 100 along with the endoscope distal end 104 within the endoscope distal tip attachment 102 thereby engaging with the endoscope 100 and the endoscope distal end 104. Moreover, the outer barrel ribs 300 can extend from the shoulder 306 towards the outer barrel proximal end 302.

As noted above, the ring 304 can assist with pulling the outer barrel ribs 300 into the configuration shown with reference to FIG. 3 after outward deflection during placement of the endoscope distal tip attachment 102 over the endoscope distal end 104. The outer barrel 200 can also include members 308 that can couple with the outer barrel ribs 300, as shown in FIG. 3. Similar to the ring 304, the member 308 can function to move the outer barrel ribs 300 to the position shown in FIG. 3 after the outer barrel ribs 300 have deflected during placement of the endoscope distal tip attachment 102 over the endoscope distal end 104. The member 308 can be formed of an elastomer, such as polyurethane, polybutadiene, neoprene and silicone, or any other type of material that allows the member 308 to pull the outer barrel ribs 300 into the configuration shown in FIG. 3 after outward deflection during placement of the endoscope distal tip attachment 102 over the endoscope distal end 104. Therefore, the member 308 can be formed of a material that is capable of expansion and contraction.

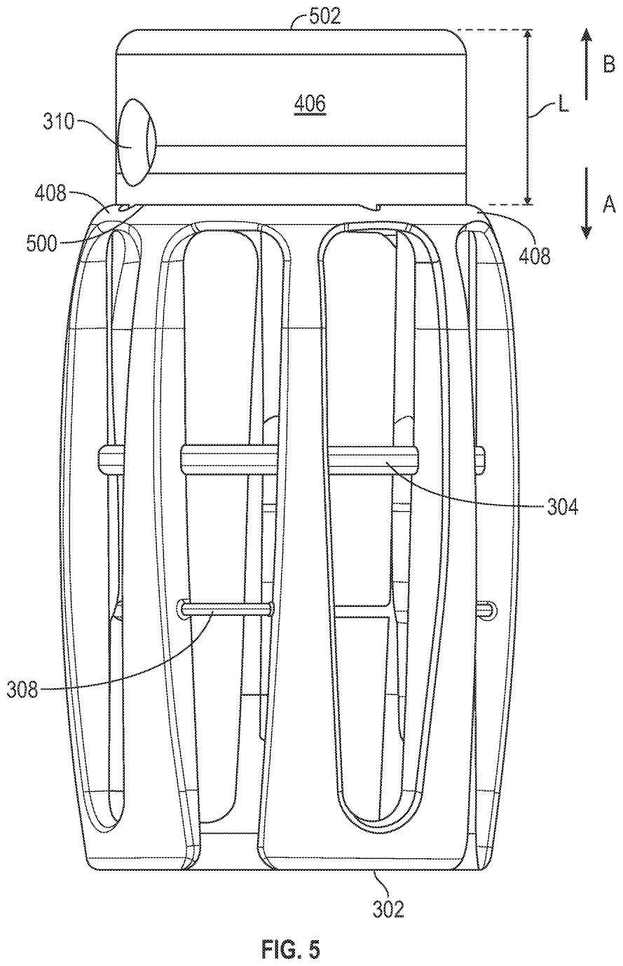

As noted above, the endoscope distal tip attachment 102 can include the distal tip 106. The distal tip 106 can be configured to help reduce or minimize foreign bodies 108 from obscuring a camera 110 of the endoscope assembly 100. In particular, FIGS. 4 and 5 show how the distal tip 106 can include a wall 404 that can define a cavity 406. The distal tip wall 404 can have a circular configuration which defines the distal tip cavity 406. Moreover, the outer barrel 200 can include a shoulder 408 disposed at a second side 500 (FIG. 5) of the outer barrel distal end 204. The shoulder 408 can oppose the shoulder 306 such that the outer barrel ribs 300 extend along a direction A from the shoulder 306 and the distal tip 106 extends along a direction B from the shoulder 408 opposite from the direction A and the outer barrel ribs 300.

The distal tip 106 can extend between a distance L from the shoulder 408. For example, the distance can be between about 8.7 mm and 11.0 mm. The distance L of the distal tip 106 can be set to inhibit or prevent the foreign bodies 108 from obscuring the endoscope distal end 104 and camera 110. In addition, the distance L of the distal tip 106 can be set to protect the endoscope distal end 104 and the camera 110 from any tissue present at a site being examined by the endoscope assembly 100. In particular, the distance L can be set according to a procedure for which the endoscope assembly 100 is being used, such as observation and/or treatment.

As noted above, the shoulder 306 can act as stop for the endoscope distal end 104. Therefore, the camera 110 of the endoscope 101 can be separated by the distance L from a distal end 502 of the distal tip 106. By virtue of the distal tip cavity 400, the endoscope distal end 104 along with the camera 110 can be exposed to an environment within which the endoscope 101 is being used. Furthermore, the distal tip cavity 406 can protect the endoscope distal end 104 and the camera 110 from the foreign bodies 108 and may help inhibit or prevent the foreign bodies 108 from obscuring the endoscope distal end 104 and camera 110. Similarly, the distal tip cavity 406 can protect the endoscope distal end 104 and the camera 110 from any tissue present at a site being examined by the endoscope assembly 100.

The distal tip 106 can also include one or more irrigant passageways 310 formed in the distal tip wall 404, such as shown in FIGS. 3-5. While two irrigant passageways 310 are shown, the distal tip 106 can include any number of irrigant passageways 310. The irrigant passageways 310 can allow for the outflow of irrigant from a surgical site being examined with the endoscope 101. Similarly, the irrigant passageways 310 can help allow for the inflow of irrigant to a surgical site being examined with the endoscope 101.

As discussed above, the distal tip 106 can be unitary with the outer barrel 200. To help achieve a unitary configuration, the distal tip 106 can be formed as a single piece with the outer barrel 200. In particular, by virtue of the distal tip 106 being unitary with the outer barrel 200, the distal tip 106 and the outer barrel 200 are a single piece. This can help avoid the problem of securing a distal tip to an outer barrel since the distal tip 106 and the outer barrel 200 are a single, unitary, piece. The distal tip 106 and the outer barrel can be formed by a single injection molding process, casting or any other process in which the distal tip 106 and the outer barrel 200 can be formed as single, unitary, unit. Moreover, the distal tip 106 can be formed from the same material as the outer barrel 200.

More specifically, similar to the outer barrel 200, the distal tip 106 can be formed from a material having a durometer shore hardness in a range of about 70-90. Types of materials that can be used can include polymers and rubbers. For example, the distal tip 106 can be formed of a material that has a higher durometer shore hardness in comparison to the material used for the inner barrel 202. Thus, the distal tip 106 can be made of a material that is harder than a material for the inner barrel 202 when the inner barrel 202 is formed having a durometer shore hardness that is less than a durometer shore hardness of the distal tip 106. Furthermore, the distal tip 106 can be formed of a clear material.

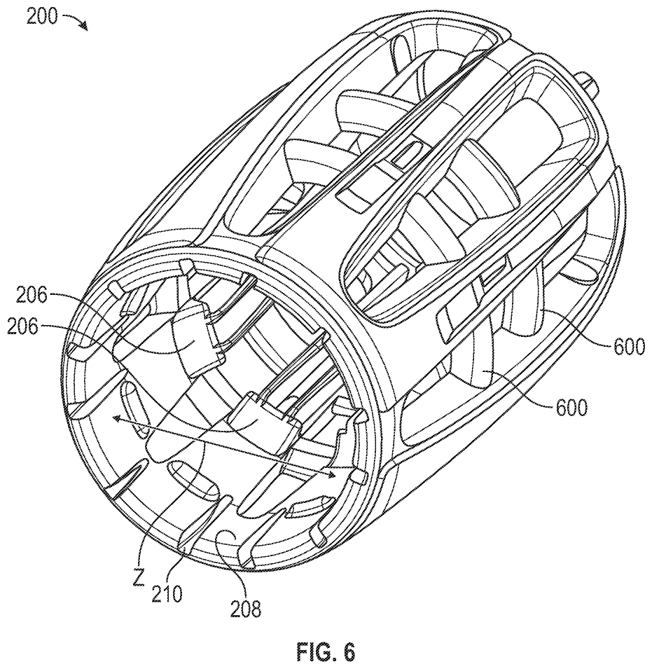

As noted above, the endoscope assembly can include the inner barrel 202. Now making reference to FIG. 6, a perspective view of the inner barrel 202 is shown. As noted above, the inner barrel 202 can include the inner barrel ribs 206 along with the engagement surfaces 208 separated by the gaps 210. The inner barrel 202 can be formed of a material that is softer than the outer barrel 200 such that the inner barrel 202 can expand and contract along a directional arrow Z relative to the outer barrel 200 when the inner barrel 202 receives the endoscope distal end 104. The inner barrel 202 can be formed of a material that is softer the material used to form the outer barrel 200 such that the inner barrel 202 can flex more than the outer barrel 200. The inner barrel 202 can be formed from a material having a durometer shore hardness in a range of about 70-90. Types of materials that can be used can include polymers and rubbers. As indicated above, the outer barrel 200 can also be formed from a material having a durometer shore hardness in a range of about 70-90. Here, the outer barrel 200 can be formed from a material having a durometer shore hardness in a range of about 70-90 that is higher than a material used to form the inner barrel 202, where the inner barrel is also formed of a material having hardness in a range of about 70-90, but is lower in the range than that used for the outer barrel 200.

The inner barrel 202 can expand and contract along the directional arrow Z to provide a snug fit around the endoscope distal end 104. The inner barrel 202 can include rings 600 which can be formed to allow expansion of the inner barrel 202 and in particular the inner barrel ribs 206 during insertion of the endoscope distal end 104 into the inner barrel 202. The inner barrel rings 600 can also contract upon receipt of the endoscope distal end 104 such that the inner barrel 202 can have a snug fit about the endoscope distal end 104.

In examples, the endoscope distal tip attachment 102 can be formed using a two-shot molding process. Here, the distal tip 106 along with the outer barrel 200 can be formed using a first molding process. Upon completion of the first molding process, the formed distal tip 106 and the outer barrel 200 can be transferred to another mold where the inner barrel 202, which again is formed from a softer material, is over molded onto an interior surface of the outer barrel 200.

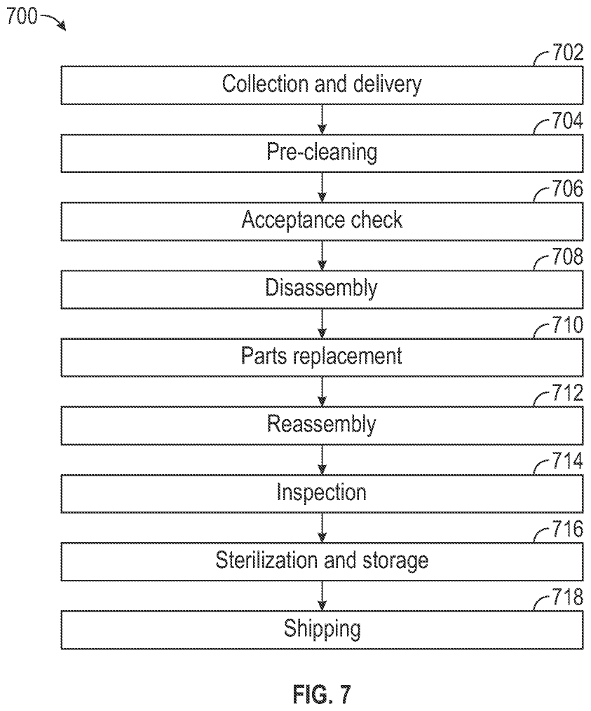

In examples, the endoscope distal tip attachment 102 can be disposed of after a single use. Alternatively, the endoscope distal tip attachment 102 can be repeatedly used a plurality of times. In examples where the endoscope distal tip attachment 102 can be repeatedly used a plurality of times, the endoscope distal tip attachment 102 can be subjected to a method 700, as shown in FIG. 7. Initially, an operator can collect the endoscope distal tip attachment 102 after it has been used for treatment and delivers that endoscope distal tip attachment 102 to a facility for treatment during an operation 702. In an operation 704, the operator can clean and sterilize the used endoscope distal tip attachment 102. Next, the operator can perform an acceptance check of the endoscope distal tip attachment 102 where the operator can determine if the endoscope distal tip attachment 102 is acceptable after cleaning and sterilization in an operation 706. Subsequently, the operator can disassemble the endoscope distal tip attachment 102 (operation 708) and, if necessary, replace various parts of the endoscope distal tip attachment 102 with new parts during an operation 710. After part replacement, the operator can assemble a new endoscope distal tip attachment 102 during the operation 712. In some examples, the operation 712 can include adding an identifier to the endoscope distal tip attachment 102 that indicates the endoscope distal tip attachment 102 has been modified from its original condition. The identifier can include a label or other marking that designates the endoscope distal tip attachment 102 as being reprocessed, refurbished or remanufactured. After reassembling the endoscope distal tip attachment 102, the operator can inspect the endoscope distal tip attachment 102 during an operation 714 and then sterilize and store the endoscope distal tip attachment 102 during an operation 716. Furthermore, the operator can ship the endoscope distal tip attachment 102 during an operation 718.

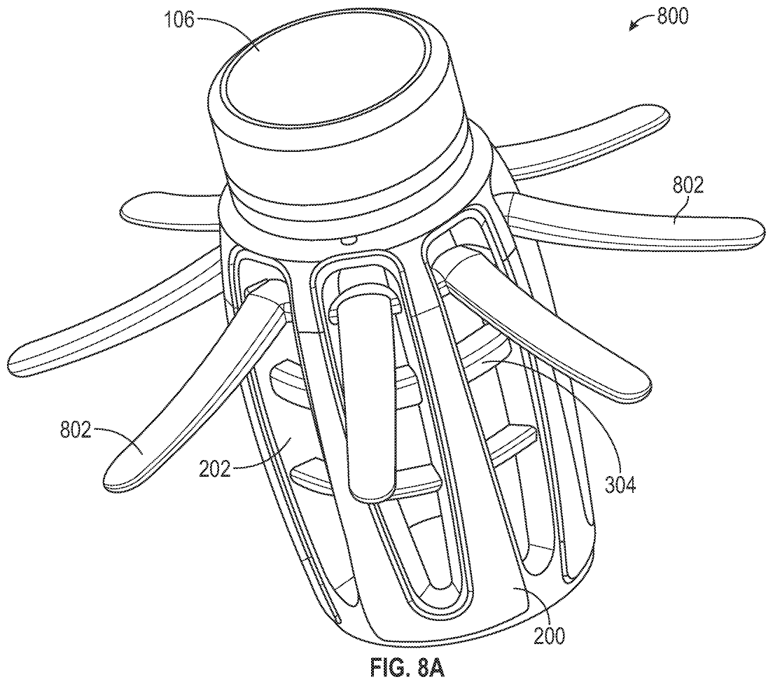

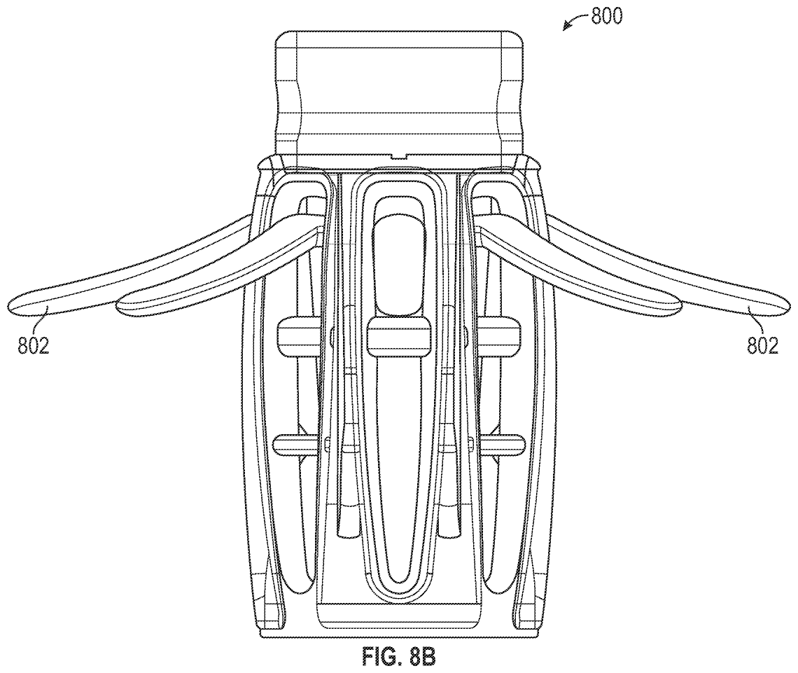

FIGS. 8A and 8B show alternative examples of a distal tip assembly attachment 800. The distal tip attachment 800 can include the outer barrel 200 and the inner barrel 202. In addition, the distal tip assembly attachment 800 can include flexible arms 802 extending therefrom. While seven arms 802 are shown, the distal tip assembly attachment 800 can any number of flexible arms 802. Moreover, the arms can be formed from the materials used to form the outer barrel 200 or the inner barrel 202 as described above. The flexible arms 802 can function to improve a view of a device that implements the distal tip assembly attachment 800. The flexible arms 802 can flatten anatomy within a target site being viewed by a device that implements the distal tip assembly attachment 800, thereby improving the view of the view of the target site.

An endoscope distal tip attachment can be situated near a distal end of an endoscope and help minimize obstructions that can interfere with the endoscope. Moreover, the endoscope distal tip attachment can include a distal tip that is unitary with an assembly that can fit over an endoscope. In examples, the distal tip only contacts a distal end of endoscope via the assembly and need not be in direct contact with the endoscope such that the distal tip does not require any type of securing to the endoscope. Furthermore, the ability to insert a distal tip over an end of an endoscope as discussed above avoids or minimizes damage to the endoscope.

The above detailed description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

What is claimed is:

1. An assembly comprising:
a barrel having a proximal end and a distal end, the barrel defining a shoulder at a first side of the distal end of the barrel, the shoulder being configured to engage with a distal end of an endoscope, the barrel including:
a set of ribs extending away from the barrel shoulder and towards the proximal end of the barrel, the set of ribs defining a cavity; and
a ring extending around the cavity defined by the set of ribs and coupled with the set of ribs; and
a distal tip extending from a second side of the distal end of the barrel that opposes the first side of distal end of the barrel and the barrel shoulder, the distal tip being formed as a unitary piece with the barrel.

2. The assembly of claim 1, further comprising an inner barrel disposed within the cavity, the barrel being formed of a first material having a first durometer shore hardness and the inner barrel being formed of a second material different from the first material and having a second durometer shore hardness that is less than the first durometer shore hardness wherein the inner barrel is configured to receive the distal end of the endoscope.

3. The assembly of claim 2, wherein the distal tip is formed of the first material having the first durometer shore hardness.

4. The assembly of claim 1, the distal tip including a wall extending from the barrel shoulder and defining a cavity through which the distal end of the endoscope is exposed.

5. The assembly of claim 4, wherein the distal tip includes an irrigant passageway formed in the wall of the distal tip.

6. The assembly of claim 1, wherein the barrel includes a second shoulder at the second side of the proximal end, the second shoulder opposing the first shoulder and wherein the distal tip extends from the second shoulder.

7. The assembly of claim 1, wherein the ring is formed in each rib of the set of ribs and the ribs of the set of ribs are configured to deflect outwardly where the ring is configured to maintain a position of the set of ribs after the ribs of the set of the ribs deflect outwardly.

8. The assembly of claim 7, wherein the ring is formed of a polymer capable of expansion and contraction.

9. An endoscope distal tip attachment comprising:
a barrel having a proximal end and a distal end, the barrel including:
a first shoulder at a first side of the distal end, the first shoulder being configured to engage with a distal end of an endoscope;
a second shoulder at second side of the distal end opposing the first side of the distal end;
a set of ribs extending away from the first shoulder and towards the proximal end of barrel, the set of ribs defining a cavity; and
a ring extending around the cavity defined by the set of ribs and coupled with the set of ribs; and
a distal tip extending from the second barrel shoulder, the distal tip being formed as a unitary piece with the barrel.

10. The endoscope distal tip attachment of claim 9, further comprising an inner barrel disposed within the cavity, the barrel being formed of a first material having a first durometer shore hardness and the inner barrel being formed of a second material different from the first material and having a second durometer shore hardness that is less than the first durometer shore hardness wherein the inner barrel is configured to receive the distal end of the endoscope.

11. The endoscope distal tip attachment of claim 10, wherein the distal tip is formed of the first material having the first durometer shore hardness.

12. The endoscope distal tip attachment of claim 9, wherein the distal tip includes a wall extending from the barrel shoulder and an irrigant passageway within the wall of the distal tip, the wall of the distal tip defining a cavity through which the distal end of the endoscope is exposed.

13. The endoscope distal tip attachment of claim 9, wherein the ring is formed in each rib of the set of ribs and the ribs of the set of ribs is configured to extend outwardly where the ring is configured to maintain a position of the set of ribs after the ribs of the set of the ribs extend outwardly.

14. The endoscope distal tip attachment of claim 13, wherein the ring is formed of a polymer capable of expansion and contraction.

15. An endoscope distal tip attachment comprising:
a barrel having a proximal end and a distal end, the barrel defining a shoulder at a first side of the distal end of the barrel, the shoulder being configured to engage with a distal end of an endoscope, the barrel including:
a set of ribs extending away from the barrel shoulder and towards the proximal end of the barrel, the set of ribs defining a cavity; and
a ring extending around the cavity defined by the set of ribs and coupled with the set of ribs; and
a distal tip extending from a second side of the distal end of the barrel that opposes the first side of distal end of the barrel and the barrel shoulder, the distal tip being formed as a unitary piece with the barrel, the distal tip including a wall extending from the barrel shoulder and defining a cavity through which the distal end of the endoscope is exposed, wherein an irrigant passageway formed in the wall of the distal tip.

16. The endoscope distal tip attachment of claim 15, wherein the ring is formed in each rib of the set of ribs and the ribs of the set of ribs is configured to extend outwardly where the ring is configured to maintain a position of the set of ribs after the rings of the set of the rings extend outwardly.

17. The endoscope distal tip attachment of claim 16, wherein the ring is formed of a polymer capable of expansion and contraction.

18. The endoscope distal tip attachment of claim 15, wherein the barrel includes a second shoulder at the second side of the proximal end, the second shoulder opposing the first shoulder and the distal tip extends from the second shoulder.

19. The endoscope distal tip attachment of claim 15, further comprising an inner barrel disposed within the cavity, the barrel being formed of a first material having a first durometer shore hardness and the inner barrel being formed of a second material different from the first material and having a second durometer shore hardness that is less than the first durometer shore hardness wherein the inner barrel is configured to receive the distal end of the endoscope.

20. The endoscope distal tip attachment of claim 19, wherein the distal tip is formed of the first material having the first durometer shore hardness.
